(19) United States
(12) Patent Application Publication
(10) Pub. No.: US 2015/0027905 A1
(43) Pub. Date: Jan. 29, 2015

(54) REAGENT COMPOSITION FOR BIOSENSORS AND BIOSENSOR COMPRISING REAGENT LAYER FORMED OF THE SAME

(71) Applicants: Jisu Kim, Seoul (KR); Gueisam Lim, Seoul (KR)

(72) Inventors: Jisu Kim, Seoul (KR); Gueisam Lim, Seoul (KR)

(73) Assignee: LG ELECTRONICS INC., Seoul (KR)

(21) Appl. No.: 14/383,044

(22) PCT Filed: Mar. 6, 2012
(86) PCT No.: PCT/KR2012/001632

§ 371 Date: Sep. 4, 2014

(51) Int. Cl. G01N 27/327; C12Q 1/00

(52) U.S. Cl. CPC G01N 27/3274; C12Q 1/004
USPC 205/777.5; 204/403.01; 204/403.14; 205/792

(57) ABSTRACT

The present invention relates to a composition which reduces the measurement error caused by the effect of hematocrit in a biosensor and to a biosensor comprising the same. Specifically, the invention relates to a reagent composition comprising an enzyme, an electron transfer mediator, a water-soluble polymer, and bile acid, and to a biosensor comprising a reagent layer formed of the composition. The reagent layer reduces the measurement error caused by the effect of hematocrit in the biosensor.

### TECHNICAL FIELD

The present invention relates to a composition capable of reducing the measurement error caused by the effect of hematocrit in a biosensor and to a biosensor comprising the same.

### BACKGROUND ART

A biosensor is a sensor which utilizes the molecule identifying abilities of biological materials such as microorganisms, enzymes and antibodies to apply the biological materials as molecular recognition elements. Specifically, the biosensor utilizes a reaction which occurs when an immobilized organic material recognizes a target specific substance, such as oxygen consumption by respiration of a microorganism, an enzyme reaction, and luminescence.

Among biosensors, enzyme sensors have been put to practical use. For example, enzyme sensors for glucose, lactic acid, cholesterol, lactose, urea, and amino acid are utilized in the medical or food industry. An enzyme sensor reduces an electron acceptor by an electron generated by a reaction between a substrate included in a sample and an enzyme, and a measuring device electrochemically measures the oxygen-reduction quantity of the electron acceptor, thereby performing quantitative analysis of the sample.

As an example of such biosensors, FIG. 1 shows an exploded perspective view of a three-electrode-type biosensor. To fabricate the biosensor shown in FIG. 1, an electrically conductive layer is formed on an electrically conductive layer 101 by a sputtering or screen printing process, and then slits are formed therein using a laser or the like, thereby forming a working electrode 102, a reference electrode and a detecting electrode 104. On these electrodes are formed a reagent layer 105 comprising an enzyme, which reacts with a specific component in a sample solution, and an electron transfer mediator. Also, on the reagent layer 105 and the electrodes 102, 103 and 104, a spacer 106 having a notch and a cover 108 are sequentially placed, thereby forming a cavity 117 to which a sample is to be supplied. Meanwhile, although supply of the sample solution from the cavity to the biosensor is realized by a capillary phenomenon, smooth supply of the sample solution is realized by providing the cover 108 with an air hole 109 for letting the air in the cavity 117 out of the biosensor.

When the sample is applied to the inlet of the cavity 117 of the biosensor thus configured, the sample is supplied from the inlet of the cavity 117 to the cavity 117 by the capillary phenomenon, and when it reaches the position of the reagent layer 1055, a specific component in the sample solution reacts with the reagent contained in the reagent layer 5. The amount of change in current which occurs due to this reaction is read with an external measurement device which is connected through the leads 110, 111 and 112 of the working electrode 102, the reference electrode 103 and the detection electrode 1044, respectively. The read current value is converted into the concentration of the specific component, thereby determining the quantity of the specific component in the sample solution.

However, the conventional biosensors have a problem in that, when a sample is blood, a correct measurement result is not obtained due to the effect of hematocrit. Particularly, the enzyme sensor for glucose determination is frequently used to measure blood glucose levels before insulin injection or to confirm low blood glucose levels, and when it indicates glucose levels higher than the correct values due to the effect of hematocrit, an excessive amount of insulin can be administered or low blood glucose levels can be passed over. For this reason, there is a need to develop a high-accuracy biosensor which is not influenced by hematocrit even when a sample is blood.

In attempts to reduce the effect of hematocrit on biosensors, the following several methods have been proposed: methods in which erythrocytes are additionally removed or a layer for removing erythrocytes is applied on a reagent layer (JP 1134461, JP 2000338076 and U.S. Pat. No. 5,658,444); a method of using a test strip comprising a screen-printable, integrated reagent/blood separation layer containing a silica filler (U.S. Pat. No. 6241862 B1); a chemometric correction method employing double excitation potentials (WO 01/57510 A2). However, these methods can require extra steps in the fabrication process or result in a large loss of the reagents in printing the reagent layer. In addition, it is difficult to simply apply the reagent mixture to the working electrode.

In another method, in order to reduce the effect of hematocrit, that is, the adsorption of erythrocytes or proteins in a sample, not only silica, but also various polymers can be applied alone or in combination to the electrode surface. However, the polymers used are generally hydrophilic, and when they come into contact with a sample, they can be dissolved with the sample, so that the network structure thereof cannot act as an effective blocker. When the polymers are non-hydrophilic, they can be repelled from the hydrophilic reagent layer, thus making it difficult to form a physiologically stable structure.

### DISCLOSURE OF INVENTION

### Technical Problem

Accordingly, the present invention has been made keeping in mind the above problems occurring in the prior art, and an object of the present invention is to provide a reagent composition, which can eliminate or reduce the bias caused by hematocrit, and a high-accuracy biosensor comprising the same.

Another object of the present invention is to provide an electrochemical biosensor which can effectively compensate for the interference of hematocrit and, at the same time, can be fabricated at low cost.

Still another object of the present invention is to provide an electrochemical biosensor which compensate for the interference of hematocrit and, at the same time, can be produced in large amounts by a simple process.

### Solution to Problem

One aspect of the present invention is a reagent composition for biosensors, comprising an enzyme, an electron transfer mediator, a water-soluble polymer, and bile acid.

Herein, the bile acid may be a hydroxyl derivative of cholic acid.

Also, the bile acid is preferably contained in an amount ranging from 1 to 50 wt %, and preferably from 5 to 30 wt %, based on the total weight of the composition.

Another aspect of the present invention is a biosensor for measuring the concentration of a specific component in a sample, wherein bile acid is contained in a reagent layer comprising a reagent which reacts specifically with the specific component in the sample.

Herein, the concentration of the specific component may be measured using electrodes, including a working electrode and a reference electrode, which are provided on an insulating layer.

The reagent layer may also comprise an enzyme and an electron transfer mediator.

Also, the reagent layer is preferably formed on the electrodes.

Also, the electrodes are preferably formed so as to be disposed in a diffusion region in which the reagent of the reagent layer is dissolved in the sample and diffused.

Also, the enzyme may be glucose oxidase.

Still another aspect of the present invention is an electrochemical biosensor comprising: a working electrode; and a reagent layer coated on the working electrode with the above-described reagent composition(That is, a reagent layer formed of the above-described reagent composition on the working electrode).

Herein, the electrochemical biosensor may further comprise a reference electrode, in which the reagent layer may be formed across the working electrode and the reference electrode.

One embodiment of the present invention is an electrochemical biosensor for measuring the concentration of a specific component in a sample containing erythrocytes, the electrochemical biosensor comprising: a substrate; a plurality of electrodes formed on the substrate; a reagent layer formed across the plurality of electrodes on the substrate and serving to react with the specific component in the sample to generate a charge corresponding to the concentration of the specific component; and bile acid for reducing the effect of hematocrit on the concentration of the specific component.

Herein, the substrate may be made of an insulating material.

Also, the plurality of electrodes include a working electrode and a reference electrode.

Still another aspect of the present invention is a method for measuring the concentration of a specific component in a sample, the method comprising the steps of: mixing the sample with bile acid to prepare a sample solution; and measuring the concentration of the specific component in the prepared sample solution using a biosensor.

Herein, the biosensor may be an electrochemical biosensor.

Also, mixing the sample with bile acid may be performed by pretreating the sample with a solution containing bile acid.

Said bile acid is preferably contained in an amount ranging from 1 to 30 wt %, and preferably from 5 to 20 wt %, based on the total weight of the sample solution.

Specific details of other embodiments are included in the following detailed description and the accompanying drawings.

### Advantageous Effects of Invention

According to the present invention, there is provided a biosensor for measuring the concentration of a specific component in a sample. In the biosensor, bile acid is contained in a reagent layer comprising a reagent which reacts specifically with the specific component in the sample, whereby a high-accuracy biosensor, on which the effect of hematocrit is reduced, can be realized. In other words, the use of the reagent layer comprising bile acid according to the present invention can reduce the measurement error caused by the effect of hematocrit in the biosensor.

Also, the present invention can provide a biosensor which makes it possible to significantly reduce the measurement error caused by the effect of hematocrit in the biosensor strip and to significantly reduce the change in signal intensity caused by electrode active materials and interfering substances which are present in blood.

### BRIEF DESCRIPTION OF DRAWINGS

FIG. 1 is a perspective view showing the configuration of a biosensor according to the prior art.

FIG. 2 is a perspective view showing the configuration of a biosensor according to one embodiment of the present invention.

FIG. 3 is a graphic diagram showing the error rates of the Example of the present invention and Comparative Examples, measured at various hematocrit levels in the Test Example of the present invention.

### DESCRIPTION OF THE REFERENCE NUMERALS IN THE DRAWINGS

101, 201, 211: substrate;

102, 203: work electrode;

103, 205: reference electrode;

104: detection electrode;

105, 207: reagent layer;

106, 209: spacer;

106a: notch;

117, 210: cavity

108: cover;

109: air hole;

110, 111, 112: lead

### BEST MODE FOR CARRYING OUT THE INVENTION

The present disclosure may have diverse modified embodiments, and thus specific embodiments are illustrated in the drawings and will now be described in detail. It is to be understood, however, that the scope of the present invention is not limited to specific embodiments and covers all the modifications, equivalents, and replacements within the idea and technical scope of the present disclosure. In the following description, the detailed description of related known technology will be omitted when it may obscure the subject matter of the present invention.

Terms used in this specification are used only for describing a specific example embodiment and are not intended to limit the scope of the present invention. Singular expressions include plural expressions unless otherwise specified in the contest thereof. In this specification, terms “include”, “have”, etc., are intended to denote the existence of mentioned characteristics, numbers, steps, operations, components, parts, or combinations thereof, but do not exclude the probability of existence or addition of one or more other characteristics, numbers, steps, operations, components, parts, or combinations thereof.

The terms “first”, “second”, etc., are used for describing various components, but the components are not limited by the terms. The terms are used only for distinguishing a component from other components.

The present invention provides a reagent composition comprising an enzyme, an electron transfer mediator, a water-soluble polymer and bile acid. The reagent composition can reduce the effect of hematocrit. Also, it can reduce the change in signal intensity caused by interfering substances, such as ascorbic acid, acetoaminophene and uric acid, which are present in blood samples.

The enzyme is reduced by reaction with various metabolites to be measured, and the reduced enzyme reacts with the electron transfer mediator, thereby quantifying the metabolites. Although the present invention is described by an example of the biosensor for measuring blood glucose levels, appropriate enzymes and electron transfer mediators can be introduced into the biosensor of the present invention, thereby quantitatively analyzing the concentrations of organic or inorganic substances in samples, including bio-materials, such as metabolites, e.g., cholesterol, lactate, creatinine, proteins, hydrogen peroxide, alcohols, amino acids, and enzymes, e.g., GPT (glutamate pyruvate transaminase), GOT (glutamate oxaloacetate transmianse), environmental samples, agricultural and industrial samples, and food samples. Thus, it is to be understood that various enzymes can be contained in the reagent composition, suggesting that the present invention can be used to quantitatively analyze various metabolites. For example, cholesterol, lactate, glutamate, hydrogen peroxide, and alcohol can be quantitatively analyzed using glucose oxidase, lactate oxidase, cholesterol oxidase, glutamate oxidase, horseradish peroxidase, or alcohol oxidase. Preferably, the enzyme that is used in the present invention may be an oxidase selected from the group consisting of glucose oxidase, glucose dehydrogenase, cholesterol oxidase, cholesterol esterase, lactate oxidase, ascorbic acid oxidase, alcohol oxidase, alcohol dehydrogenase, bilirubin oxidase, and sugar dehydrogenase.

In an embodiment of the present invention, the enzyme that is used in the biosensor for measuring blood glucose levels is preferably glucose oxidase or glucose dehydrogenase.

The electron transfer mediator reacts with a metabolite and is reduced by reaction with the reduced enzyme, and the electron transfer mediator thus reduced diffuses to the electrode surface so that it applies oxidation potential to the electrode surface, thereby generating an electric current. The electron transfer mediator that is used in the present invention may be selected from among ferrocene, ferrocene derivatives, quinones, quinine derivatives, organic conducting salts and viologen. Preferably, the electron transfer mediator that is used in the present invention may be a mixed-valence compound selected from the group consisting of hexaammineruthenium (III) chloride, potassium ferricyanide, potassium ferrocyanide, dimethylferrocene (DMF), ferricinium, ferocene monocarboxylic acid (FCOOH), 7,7,8,8-tetracyanoquino-dimethane (TCNQ), tetrathiafulvalene (TTF), nickelocene (Nc), N-methyl acidinium (NMA+), tetrathiatetracene (TTT), N-methylphenazinium (NMP+), hydroquinone, 3-dimethylaminobenzoic acid (MBTHDMAB), 3-methyl-2-benzothiozolinonehydrazone, 4-aminoantipyrin (AAP), dimethylaniline, 4-aminoantipyrene, 4-methoxynaphthol, 3,3′,5,5′-tetramethyl benzidine (TMB), 2,2-azino-di-[3-ethyl-benzthiazoline sulfonate]), o-odianisidine, o-toluidine, 2,4-dichlorophenol, 4-amino phenazone, benzidine, and prussian blue.

In the present invention, the most preferable electron transfer mediator is hexaammineruthenium (III) chloride which has the following characteristics: 1) the oxidation-reduction state thereof in aqueous solution is stable and reversible; (2) the reduced electron transfer mediator does not react with oxygen; (3) its formal potential is low enough to minimize the influence of interfering substances such as ascorbic acid, acetaminophen and uric acid; (4) the oxidation of the reduced electron transfer mediator is not sensitive to pH; and (5) it does not react with electrochemically interfering materials, such as ascorbic acid, acetaminophen and uric acid.

The water-soluble polymer serves as a support for the reagent composition and assists in the stabilization and dispersion of the enzyme. It is contained in an amount of 0.1 to 10 wt % based on the weight of the solid-state reagent composition (as measured before the composition is not dissolved in PBS buffer). The water-soluble polymer that is used in the present invention is polyvinyl pyrrolidone (PVP), polyvinyl alcohol (PVA), perfluorosulfonate, hydroxyethyl cellulose (HEC), hydroxypropyl cellulose (HPC), carboxy methyl cellulose (CMC), cellulose acetate or polyamide. Most preferably, it is PVP or HPC.

In addition, the reagent contains a substance which can significantly reduce the measurement error caused by the effect of hematocrit.

The present invention is characterized by using bile acid. Bile acid is added to the reagent composition, whereby it reduces the bias attributable to hematocrit. Also, it reduces the linear dynamic range of the biosensor at high hematocrit levels. Bile acid is dissolved in water or a water-miscible liquid before being added to the composition. The bile acid is added to the solid-state composition in an amount of 0.1 to 50 wt %, more preferably 1 to 50 wt %, and most preferably 5 to 30 wt %. If bile acid is added in an amount lower than the lower limit of the above-mentioned range, it will be ineffective, and if it is added in an amount of more than 50 wt %, it will not be sufficiently dissolved. Bile acid that is used in the present invention is a kind of steroid compound is a major solid component of the bile of vertebrates, which is weakly acidic. Bile acids from the upper vertebrates such as mammals are mostly hydroxy derivatives of cholic acid. Human bile contains cholic acid, deoxycholic acid, chenodeoxycholic acid, tricholic acid and the like. The kind and composition of bile acid slightly vary depending on the kind of animal. Bile is composed of bile acids conjugated with glycine or taurine.

The present invention also provides an electrochemical biosensor. Specifically, the present invention provides an electrochemical biosensor comprising a reagent layer formed of the inventive composition on a working electrode. The reagent composition may be a sensing layer composition, and the reagent layer formed of the composition may be a sensing layer.

The electrochemical biosensor of the present invention comprises: a base substrate and a cover; a polymer film comprising a conductive material (a carbon or metal material or a conductive polymer) and formed on the surface of each of the base substrate and the cover by screen printing or vapor deposition; a sample introduction portion provided between the base substrate and the cover; and a reagent layer serving to fundamentally reduce the bias attributable to hematocrit levels.

Also, the electrode system of the electrochemical biosensor may be formed either on the base substrate or both the base substrate and the cover. In other words, the working electrode or the reference electrode (or auxiliary electrode) may be formed on (1) the base substrate or (2) on the base substrate and the cover, respectively. In the case of (2) above, the electrodes are formed to face each other.

In the present invention, another electrode in addition to the above-described electrodes may be formed and is hereinafter referred to as the “flow-sensing electrode”. The flow-sensing electrode is disposed behind the working electrode and serves to measure the fluidity of a whole blood sample on the base substrate. Because hematocrit changes the fluidity of whole blood and the electrical conductivity of blood, the time required to fill the sample introduction portion with blood through the elongated capillary channel of the biosensor changes in proportion to hematocrit. This change in the fluidity of the blood sample can be sensed using the flow-sensing electrode, and thus the flow-sensing electrode can be used to correct the error caused by hematocrit in the measurement of blood glucose levels.

The reagent agent that is used as a sensing layer in the present invention can be formed by simply applying the ragent composition to the working electrode. Also, the biosensor of the present invention may further comprise a reference electrode, and the reagent layer may be formed across the working electrode and the reference electrode. Herein, the amount of the reagent composition is preferably 300 to 500 nl. The reagent composition that is used in the present invention comprises an enzyme, an electron transfer mediator, a water-soluble polymer and bile acid and can reduce the effect of hematocrit. In addition, the reagent composition can reduce the change in signal intensity caused by interfering substances, such as ascorbic acid, acetoaminophene and uric acid, which are present in blood.

FIG. 2 is an exploded perspective view showing a test strip for electrochemical biosensors according to the present invention.

The present invention provides a biosensor for measuring the concentration of a specific component in a sample, wherein bile acid is contained in a reagent layer 207 comprising a reagent that reacts with the specific component in the sample.

Hereinafter, the present invention will be described by example of an enzyme sensor that uses an enzyme as a molecule recognition element that reacts specifically with a specific component in a sample.

As shown in FIG. 2, a test strip 200 for electrochemical biosensors comprises: an insulating substrate 201; electrodes 203 and 205 formed on the insulating substrate 201; and a reagent layer 207 formed across the electrodes 203 and 205 on the insulating substrate 201, the reagent layer 207 serving to react with a specific component in a sample to generate a charge corresponding to the concentration of the specific component. Thus, the concentration of the specific component is measured using the working electrode 203 and the reference electrode 205, which are formed on the substrate 201. Also, the reagent layer 207 comprises bile acid for reducing the effect of hematocrit on the concentration of the specific component.

In FIG. 2, the insulating substrate 201 on which the working electrode 203 and the reference electrode 205 are formed may be attached to another insulating substrate 209 with spacers 209 interposed therebetween. A cavity 210 formed between the spacers 209 provides a channel for introducing a sample. Although FIG. 2 illustrates the two-electrode system, the present invention can, of course, be applied to a three-electrode system.

As shown in FIG. 2, the reagent layer 207 is formed on the electrodes 203 and 205 and is preferably fixed across the working electrode 203 and the reference electrode 205. The reagent layer 207 can be formed on a portion of the insulating substrate 201, which corresponds to the cavity 210, using various methods, including an automated dispenser, screen printing, roll coating, spin coating, etc. Also, the electrodes 203 and 205 may be formed so as to be disposed in a diffusion area in which the reagent of the reagent layer 207 is dissolved in the sample and diffused. By doing so, when a physiological sample is introduced, the reagent layer 207 will react with the sample to generate a charge, and when a suitable voltage is applied between the two electrodes 203 and 205, an electric current corresponding to the concentration of an analyte in the sample will flow between the electrodes 203 and 205.

The reagent layer 207 may comprise: a biochemical substance (e.g., enzyme, antibody, protein, etc) having the ability to recognize a molecule; an electrode transfer mediator capable of effectively transferring a charge, produced by a biochemical reaction, to the electrode surface; a hydrophilic polymer compound serving as a support between the electrode surface and the biochemical substance; and a surfactant serving as a dispersing agent. The enzyme that is used in the present invention varies depending on a substance to be detected, and for example, if glucose is to be detected, glucose oxidase may be used as the enzyme.

One embodiment of the present invention is an electrochemical biosensor for measuring the concentration of a specific component in a sample containing erythrocytes, the electrochemical biosensor comprising: a substrate 201; a plurality of electrodes 203 and 205 formed on the substrate; a reagent layer formed across the plurality of electrodes on the substrate and serving to react with a specific component in the sample to generate a charge corresponding to the concentration of the specific component; a bile acid for reducing the effect of hematocrit on the concentration of the specific component.

The substrate 201 may be made of a ceramic, glass or polymer material. Preferably, it may be made of an organic polymer material, such as polyester, polyvinyl chloride or polycarbonate. In addition, it may be made of an insulating material.

The plurality of electrodes may include the working electrode 203 and the reference electrode 205 and may further include an auxiliary electrode. The material of the working electrode 203, the reference electrode 205 and the auxiliary electrode is not specifically limited, so long as it is a conductive material. Examples of the conductive material include silver, epoxy resin, palladium, copper, gold, platinum, iridium, silver/silver chloride, carbon reinforced with a redox pair or other additives, etc.

Another aspect of the present invention is a method for measuring the concentration of a specific component in a sample, the method comprising the steps of: mixing a sample with bile acid to prepare a sample solution; and measuring the concentration of a specific component in the prepared sample solution using a biosensor. Herein, the biosensor may be a general biosensor, particularly a biosensor employing an electrochemical method. Preferably, it is the above-described biosensor of the present invention.

The present invention is characterized in that bile acid is used to reduce the effect of hematocrit. Thus, in the present invention, bile acid may be allowed to react with a sample. In addition, the sample may also be pretreated with a solution containing bile acid. Then, bile acid that is used in the present invention can minimize the effect of hematocrit on the concentration o a specific component in the sample.

Herein, the bile acid is preferably contained in an amount ranging from 1 to 30 wt %, and more preferably from 5 to 20 wt %, based on the total weight of the sample solution. If the bile acid is added in an amount lower than the lower limit of the above-mentioned range, it will be ineffective, and if it is added in an amount of more than 30 wt %, it will not be easily dissolved.

### Mode for the Invention

Hereinafter, the present invention will be described in detail with reference to examples. It is to be understood, however, that these examples are for illustrative purposes only and are not intended to limit the scope of the present invention.

### EXAMPLE

### Biosensor Comprising a Reagent Layer Including Bile Acid

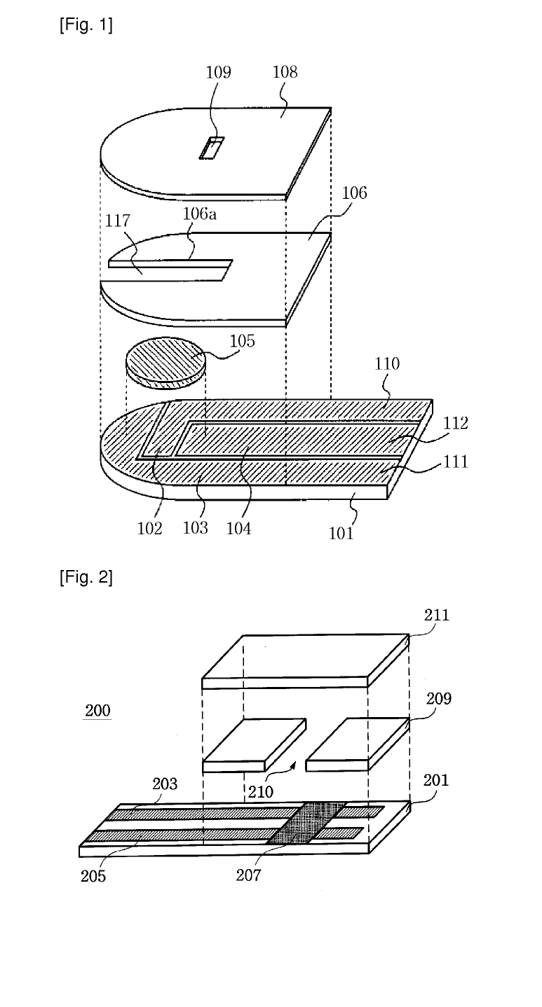

In this Example, a biosensor was fabricated by adding bile acid to the reagent layer 105 of the biosensor shown in FIG. 1. Herein, glucose oxidase was used as an enzyme.

### Comparative Examples

### Electrochemical Biosensors According to the Prior Art

As biochemical devices employing electrochemistry, a plurality of biosensors which have been frequently used to measure blood glucose levels were prepared.

Specifically, the following biosensors were purchased: a biosensor (Roche; Comparative Example 1) known to carry out correction for hematocrit (Hct) by a mechanical method of measuring the flow rate of blood in the sensor or measuring the density of the density of blood on the electrode; a biosensor (Onetouch; Comparative Example 2); and a biosensor (I-sens; Comparative Example 3) known to carry out correction for hematocrit by a chemical method using fatty acid and quaternary ammonium.

In addition, as a control, the biosensor as described in the above Example, in which no bile acid was added to the reagent layer, was prepared (Comparative Example 4).

### Test Example

### Comparative Analysis of Blood Glucose Levels

Vein blood was collected from a plurality of patients, and the glucose levels of the blood were measured using YSI (large-sized blood glucose measurement system for hospitals approved by the FDA, followed by separation of red blood cells (RBCs) and plasma.

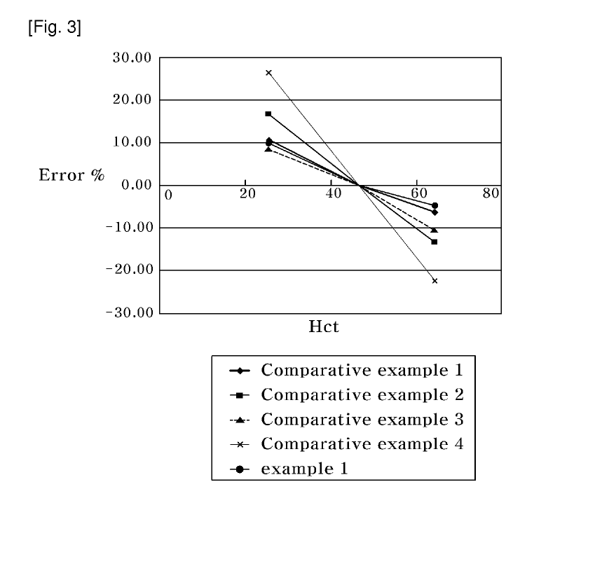

Based on the ISO standards, the blood was diluted to various concentrations (500 uL, 200 uL and 100 uL for RBCs; 200 uL for plasma). Then, the blood glucose levels of the diluted blood were measured using each of the biosensors prepared in the above Example and Comparative Examples, and the measurement results are shown in Tables 1 to 6 and FIG. 3.

TABLE 1
Hct of each sample

| Whole | Red Blood | |
|---|---|---|
| blood | cells | Hct |
| 2.8 | 1.8 | 64.29 |
| 2.8 | 1.3 | 46.43 |
| 2.95 | 0.75 | 25.42 |

TABLE 2
For Hct of 64.29

| | Comp. | Comp. | Comp. | Comp. | |
|---|---|---|---|---|---|
| | Example | Example | Example | Example | |
| Strip | 1 | 2 | 3 | 4 | Example |
| 1 | 158 | 129 | 146 | 121 | 143 |
| 2 | 156 | 133 | 148 | 119 | 153 |
| 3 | 150 | 139 | 142 | 124 | 151 |
| 4 | 159 | 142 | 143 | 111 | 149 |
| 5 | 155 | 139 | 151 | 116 | 154 |
| Average | 155.6 | 136.4 | 146.0 | 118.2 | 150.0 |
| SD | 3.5 | 5.3 | 3.7 | 5.0 | 4.4 |
| CV | 2.3 | 3.9 | 2.5 | 4.2 | 2.9 |

TABLE 3
For Hct of 46.43

| | Comp. | Comp. | Comp. | Comp. | |
|---|---|---|---|---|---|
| | Example | Example | Example | Example | |
| Strip | 1 | 2 | 3 | 4 | Example |
| 1 | 166 | 158 | 164 | 152 | 158 |
| 2 | 165 | 156 | 166 | 156 | 162 |
| 3 | 169 | 162 | 162 | 148 | 153 |
| 4 | 158 | 151 | 169 | 151 | 156 |
| 5 | 171 | 159 | 153 | 155 | 157 |
| Average | 165.8 | 157.2 | 162.8 | 152.4 | 157.2 |
| SD | 5.0 | 4.1 | 6.1 | 3.2 | 3.3 |
| CV | 3.0 | 2.6 | 3.7 | 2.1 | 2.1 |

TABLE 4
For Hct of 25.42

| | Comp. | Comp. | Comp. | Comp. | |
|---|---|---|---|---|---|
| | Example | Example | Example | Example | |
| Strip | 1 | 2 | 3 | 4 | Example |
| 1 | 182 | 182 | 174 | 193 | 172 |
| 2 | 183 | 184 | 181 | 192 | 173 |
| 3 | 188 | 189 | 179 | 199 | 176 |
| 4 | 179 | 172 | 177 | 186 | 169 |
| 5 | 186 | 191 | 173 | 194 | 175 |
| Average | 183.6 | 183.6 | 176.8 | 192.8 | 173.0 |
| SD | 3.5 | 7.4 | 3.3 | 4.7 | 2.7 |
| CV | 1.9 | 4.1 | 1.9 | 2.4 | 1.6 |

TABLE 5
Average of blood glucose levels

| | Comp. | Comp. | Comp. | Comp. | |
|---|---|---|---|---|---|
| | Example | Example | Example | Example | |
| Strip No. | 1 | 2 | 3 | 4 | Example |
| 64.29 | 155.6 | 136.4 | 146.0 | 118.2 | 150.0 |
| 46.43 | 165.8 | 157.2 | 162.8 | 152.4 | 157.2 |
| 25.42 | 183.6 | 183.6 | 176.8 | 192.8 | 173.0 |

TABLE 6
Error rate(%)

| | Comp. | Comp. | Comp. | Comp. | |
|---|---|---|---|---|---|
| | Example | Example | Example | Example | |
| Strip No. | 1 | 2 | 3 | 4 | Example |
| 64.29 | −6.15 | −13.23 | −10.32 | −22.44 | −4.58 |
| 46.43 | 0 | 0 | 0 | 0 | 0 |
| 25.42 | 10.74 | 16.79 | 8.60 | 26.51 | 10.05 |

FIG. 3 is a graphic diagram showing the error rates of the Example and the Comparative Examples, measured at various hematocrit levels in the Test Example of the present invention. As shown therein, the effect of Hct on the biosensors at high Hct levels was the lowest in the biosensor of the Example. In addition, the biosensor of the Example had a significantly excellent effect even at low Hct levels.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A reagent composition for biosensors, comprising an enzyme, an electron transfer mediator, a water-soluble polymer, and bile acid.

2. The reagent composition of claim 1, wherein the bile acid is a hydroxyl derivative of cholic acid.

3. The reagent composition of claim 1, wherein the the bile acid is contained in an amount ranging from 1 to 50 wt % based on the total weight of the composition.

4. The reagent composition of claim 1, wherein the the bile acid is contained in an amount ranging from 5 to 30 wt % based on the total weight of the composition.

5. A a biosensor for measuring the concentration of a specific component in a sample, wherein bile acid is contained in a reagent layer comprising a reagent which reacts specifically with the specific component in the sample.

6. The biosensor of claim 5, wherein the concentration of the specific component is measured using electrodes, including a working electrode and a reference electrode, which are provided on an insulating layer.

7. The biosensor of claim 6, wherein the reagent layer comprises an enzyme and an electron transfer mediator.

8. The biosensor of claim 7, wherein the reagent layer is formed on the electrodes.

9. The biosensor of claim 7, wherein the electrodes are formed so as to be disposed in a diffusion area in which the reagent of the reagent layer is dissolved in the sample and diffused.

10. The biosensor of claim 7, wherein the enzyme is glucose oxidase.

11. An electrochemical biosensor comprising:
a working electrode; and
a reagent layer coated on the working electrode with the reagent composition of claim 1.

12. The electrochemical biosensor of claim 11, further comprising a reference electrode, in which the reagent layer is formed across the working electrode and the reference electrode.

13. An electrochemical biosensor for measuring the concentration of a specific component in a sample containing erythrocytes, the electrochemical biosensor comprising:
a substrate;
a plurality of electrodes formed on the substrate;
a reagent layer formed across the plurality of electrodes on the substrate and serving to react with the specific component in the sample to generate a charge corresponding to the concentration of the specific component; and
bile acid for reducing the effect of hematocrit on the concentration of the specific component.

14. The electrochemical biosensor of claim 13, wherein the substrate is made of an insulating material.

15. The electrochemical biosensor of claim 13, wherein the plurality of electrodes include a working electrode and a reference electrode.

16. A method for measuring the concentration of a specific component in a sample, the method comprising the steps of:
mixing the sample with bile acid to prepare a sample solution; and
measuring the concentration of the specific component in the prepared sample solution using a biosensor.

17. The method of claim 16, wherein the biosensor is an electrochemical biosensor.

18. The method of claim 16, wherein mixing the sample with bile acid is performed by pretreating the sample with a solution containing bile acid.

19. The method of claim 16, wherein the bile acid is contained in an amount ranging from 1 to 30 wt % based on the total weight of the sample solution.

20. The method of claim 16, wherein the bile acid is contained in an amount ranging from 5 to 20 wt % based on the total weight of the sample solution.
